(19) United States
(12) Patent Application Publication
(10) Pub. No.: US 2016/0113753 A1
(43) Pub. Date: Apr. 28, 2016

(54) IMPLANTABLE MEDICAL DEVICE EXHIBITING DIMINISHING RADIAL FORCE

(71) Applicant: COOK MEDICAL TECHNOLOGIES LLC, BLOOMINGTON, IN (US)

(72) Inventor: KASPER KLAUSEN, Lille Skensved (DK)

(73) Assignee: Cook Medical Technologies LLC

(21) Appl. No.: 14/921,521

(22) Filed: Oct. 23, 2015

(30) Foreign Application Priority Data
Oct. 23, 2014 (GB) 1418873.4

(51) Int. Cl. A61F 2/01

(52) U.S. Cl. CPC A61F 2/013; A61F 2002/016; A61F 2210/0004; A61F 2250/0031; A61F 2250/0018; A61F 2250/0029; A61F 2310/00011

(57) ABSTRACT

A filter assembly includes a plurality of leg elements in two sets which have a generally conical form and are held together at a coupling element or hub. Disposed by the hub is a biodegradable fastener element into which proximal ends of the leg elements are embedded. The fastener element is substantially rigid and has the effect of reducing the lengths (L₂, L₃) of the legs of the leg elements which is able to flex, contributing to the generation of a greater radial force by the filter assembly. The fastener element will degrade over a given period, such as two to three weeks, thereby allowing flexing of the proximal ends of the leg elements embedded within the fastener element, resulting in an increase in the length of the leg elements able to flex and as a consequence a reduction in the radial force generated against the vessel wall.

### CROSS-REFERENCE TO RELATED APPLICATIONS

This application claims the benefit of priority under 35 U.S.C. §119(a) to Great Britain Patent Application No. GB 1418873.4, filed Oct. 23, 2014, which is hereby incorporated by reference in its entirety.

### TECHNICAL FIELD

The present invention relates to an implantable medical device such as a vascular filter, optionally for implantation in the inferior vena cava. The teachings herein are applicable also to other types of medical device, for instance to vascular occluders and the like.

### BACKGROUND ART

Implantable medical devices are being increasingly adopted for treating a variety of vascular and circulatory conditions. There are many varieties of implantable medical devices including for example stents, stent grafts, filters, occluders, prostheses and so on. Many of these devices are designed for endoluminal delivery from a remote percutaneous entry point, typically using the Seldinger technique.

A characteristic requirement of such devices is that they need to be securely implanted in a vessel so as to avoid migration of the device as a result of blood pressure or vessel movement. Implantable devices are often provided with anchoring elements, usually in the form of barbs, attached to the extremities of at least some of the elements of the device, which anchor into the vessel wall. For this to be achieved reliably, it is necessary for the device to press against the vessel wall. While this can reliably fix the device in the vessel, there can be long term problems including ballooning of the vessel at the pressure points generated by the medical device, as well as continued penetration into the vessel wall tissue of the anchor elements and parts of the device attached thereto, leading in some cases to perforation through the entire thickness of the vessel wall.

While some attempts have been made to seek to mitigate the above mentioned difficulties, these have not been entirely successful for a variety of reasons.

Some examples of prior art vascular filters and other medical devices are disclosed in US-2009/0306703, US-2012/0143238, US-2012/0029614, US-2010/0185230, US-2010/0042135, US-2007/0173885, US-2007/0112372, US-2005/0107822, US-2003/0176888, US-2012/0245620, US-2012/0083823, US-2003/0208227, US-2008/0027481, US-2009/0012596 and US-2004/0098095.

### DISCLOSURE OF THE INVENTION

The present invention seeks to provide an improved implantable medical device. The device may be a vascular filter such as a vena cava filter, an occluder or other medical device.

According to an aspect of the present invention, there is provided an implantable medical device including: a plurality of leg elements made of flexible material, each leg element including a first end connected to the coupling member and a second end remote from the coupling member, each leg element having a length between the coupling member and its second end, at least the first end of the leg elements extending in a deployed configuration from the coupling member; and a biodegradable fastener element, the plurality of leg elements being fixed in position in the fastener element along a portion of the leg elements adjacent the first end of the leg elements, wherein the fastener element substantially fixes said portions of the legs in the deployed configuration.

This structure provides a mechanism able to reduce the radial force generated by the legs of the medical device a period after implantation of the device, typically after degradation of the fastener element. Typically, the medical device needs to generate the greatest holding force when first implanted. The extremities of the legs will, though, become embedded into the vessel wall as a result of endothelialisation, which will provide an additional anchoring effect. Once so embedded, it is not necessary for the device per se to generate the same pressure against the vessel wall as is required on first implantation. Thus, the structure provides for a reduction in the radial expansion force generated by the medical device after a period, thereby in order to avoid or at least reduce incidence of ballooning of the vessel and to avoid or reduce the risk of continued penetration of the legs into the vessel wall. Moreover, the structure ensures that the medical device does not change its shape or configuration between the time when it is first implanted and after degradation of the fastener element. Thus, the shape of the medical device remains steady and reliable in all its configurations.

In one embodiment the leg elements are embedded in the fastener element while in another embodiment the fastener element has a plurality of recesses within which the leg elements are held.

Advantageously, the leg elements are fixed in the fastener element with substantially no tension applied by the fastener element to the leg elements.

In an embodiment, the fastener element is disposed adjacent the coupling member, advantageously in abutment with the coupling member. Preferably, the fastener element is attached to or integral with the coupling member.

In the preferred embodiment, the fastener element is substantially rigid. The fastener element has a resiliency less than a resiliency of the leg elements. Preferably, the fastener element reduces or prevents flexure of the portions of the leg elements embedded in the fastener element. This in effect reduces the length of the leg elements able to flex until after degradation of the fastener element. Thus, degradation of the fastener element preferably causes an increase in flexure length of the leg elements.

In an embodiment, the fastener element has a length at least 10 to 20% of the length of the leg elements. The fastener element may have a length in the region of 5 mm to 10 mm for a medical device having leg elements of a length of around 50 mm.

Advantageously, the leg elements retain substantially the same non-biased configuration when partially embedded in the fastener element as on degradation of the fastener element.

In the preferred embodiment, the first ends of the legs are rigidly attached to the coupling member, thereby able to continue to apply a radial expansion force after degradation of the fastening element.

The device may be a filter, such as a vena cava filter.

The legs may extend radially outwardly of the coupling member in a generally conical form.

Other features and advantages of the teachings herein will become apparent from the following description of the preferred embodiments.

### BRIEF DESCRIPTION OF THE DRAWINGS

Embodiments of the present invention are described below, by way of example only, with reference to the accompanying drawings, in which:

FIG. 1 is a schematic diagram of an embodiment of vascular filter having a bioabsorbable fastener element attached thereto;

FIG. 2 is a schematic diagram in partial cross-section showing a part of the filter of FIG. 1;

FIG. 3 is a schematic cross-sectional view of the fastener element of FIGS. 1 and 2;

FIG. 4 is a schematic diagram of the vascular filter of FIG. 1 after degradation of the bioabsorbable fastener element; and

FIG. 5 is a schematic cross-sectional view of another embodiment of bioabsorbable fastener element.

### DESCRIPTION OF THE PREFERRED EMBODIMENTS

It is to be understood that the drawings are schematic only and do not show the elements in proportion. The skilled person will readily appreciate the typical dimensions and proportions of the various elements depicted and will also know that these will also vary in dependence upon the nature of the vessel in which the device is to be implanted.

Although the preferred embodiments are directed to a vascular filter, such as a vena cava filter, the teachings herein are applicable to other medical devices including, for example, occluders and the like. The teachings can be used in any medical device for which it is desired to have the device exert different, particularly reducing, forces over a period of time. The described embodiments are to a conical shape filter, though the teachings herein apply equally to medical devices having other shapes including, for example, double structures having a double cone or hourglass shape.

The term “flexure length” used herein is intended to relate to the length of the leg elements able to flex in use, which, as will be apparent from the disclosure herein, will be dependent upon the state of the fastener element.

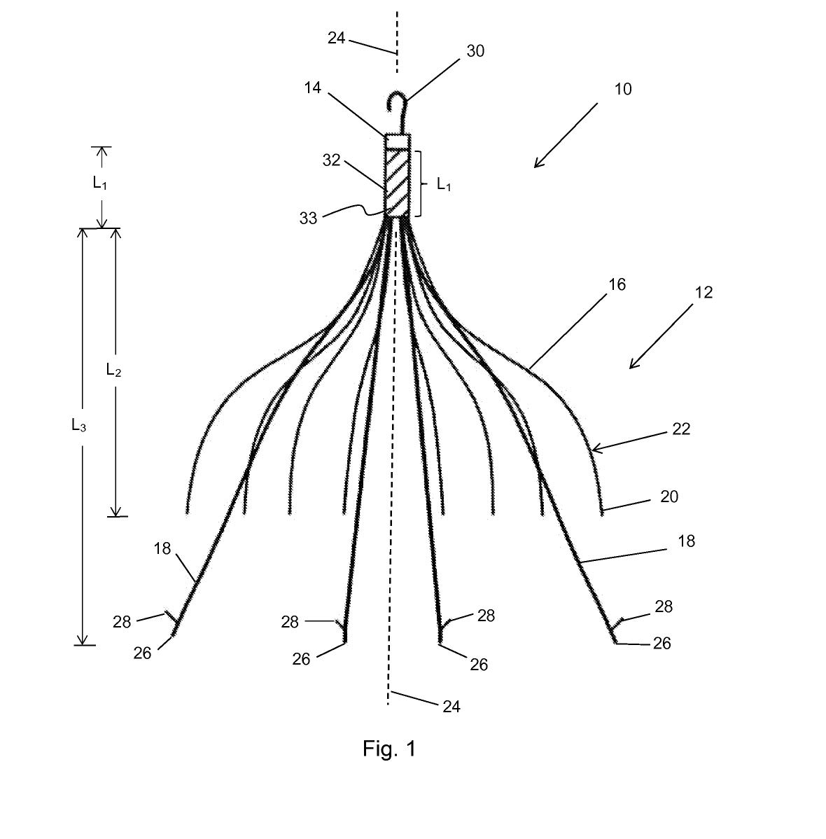

Referring first to FIG. 1, this shows an embodiment of implantable vascular filter 10 provided with a plurality of filter leg elements 12 extending from a coupling member or hub 14. In this embodiment, though not necessary in all embodiments, the leg elements 12 include two sets of legs, a first set of shorter legs 16 and a second set of longer legs 18. The leg elements 12 are formed of a resilient material, for example spring steel, a cobalt based alloy such as Elgiloy™, a shape memory alloy such as Nitinol and the like. The leg elements 12 are therefore able to flex when pressure is applied to them and when flex will generate an opposing force as a result of their resiliency.

The legs 16 of the first set have a first end (not shown in FIG. 1) which is attached to the hub 14 and a second, remote, end 20. The legs 16 have a generally curved shape, curving outwardly from their first ends and then in reverse curvature, that is radially inwardly, thereafter so as to present an inwardly curving outer surface 22. At least one portion of the legs 16 has a maximum distance from a centreline 24 of the filter 10 and which in practice will come into abutment with the inner surface of a vessel wall. The inwardly curved portion of the legs 16 may have a greater curvature than that shown in FIG. 1, such that the distal ends 20 of the legs 16 are radially closer to the centreline 24 of the filter and such that the point of maximum radial distance is at an intermediate position along the length of the legs 16.

The plurality of legs 16 are disposed in a radially spaced arrangement around the hub 14 so as to have a generally conical shape extending around the circumference of the filter 10.

In practice, the legs 16, are made to have an unbiased maximum diameter which is chosen to be greater than the diameter of a vessel into which the filter 10 is designed to be deployed. As a result, the legs 16 will press against the vessel wall when the filter 10 is deployed.

The legs 18 of the second set of legs are, in the embodiment shown in FIG. 1, substantially straight and have a first end (not shown in FIG. 1) which is connected to the hub 40 and a second end 26 remote from the hub 14. The legs 18 extend to a distance from the hub 14 further than that of the legs 16 of the first set. The distal second ends 26 of the legs 18, in this embodiment, are the points of maximum radial distance of the legs 18 from the centre line 24 of the filter assembly 10. Proximate the ends 26 of the legs 18 are barbs or other anchoring elements 28, of conventional form. The legs 18 are, in a manner similar to the legs 16 of the first set, in a radially spaced arrangement around the hub 14 so as to have a generally conical shape extending around the circumference filter 10. Similarly, the distal ends 26 of the legs 18 have an unbiased maximum diameter which is chosen to be greater than the diameter of the vessel into which the filter 10 is designed to be deployed. As the result, in use, the legs 18 will be pressed inwardly when the filter assembly 10 is exposed to the vessel, thereby to generate a filter opening force against the inner walls of the vessel, this being described in further detail below.

The legs 16 and 18 of the leg elements 12 may typically fit within a lumen (not shown) of the hub 14 and be fixed thereto in any suitable manner, for instance by welding, bonding, a friction fit and so on. The hub 14 may also be provided with a hook 30 or other filter retrieval device, of a type well known in the art.

The filter assembly 10 also includes a fastener element 32 which is disposed at the coupling member or hub end 14 of the filter assembly 10, that is at the narrow end of the assembly. The fastener element 32 is made of a biodegradable material such as a bioabsorbable metal or metal alloy or a bioabsorbable polymer. Examples include polylactide (PTA) and purified terephthalic acid (PTA) polymers and co-polymers; as well as magnesium, zinc and iron based alloys and metals. The fastener element 32 has a length L₁ as depicted in FIG. 1.

The fastening element 32 preferably has a length in the region of at least 10-20% of the length of the leg elements. The fastener element may have a length in the region of 5 mm to 10 mm for a medical device having leg elements of a length of around 50 mm.

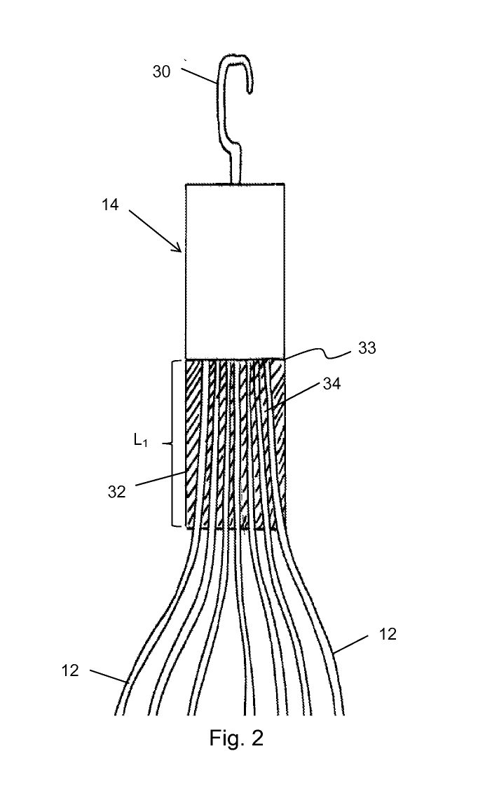

With reference to FIG. 2, this shows schematically a cross-sectional view of the fastener element 32 of the filter assembly 10 of FIG. 1 (the coupling member or hub 14 and hook 30 being depicted in full). It is to be understood that FIG. 2 is schematic only and is provided simply to depict the structure within the fastener element 32. As can be seen, in this embodiment the fastener element 32 is of solid material throughout its volume and has embedded therewithin the first or proximal ends of the legs 16 and 18 of the leg elements 12. It is preferred that the proximal ends 34 of the leg elements are embedded within the fastener element 32 in their deployed configurations, that is in their arrangement and orientations that they would have when the filter assembly 10 is expanded in the manner shown in FIG. 1 and also in FIG. 4.

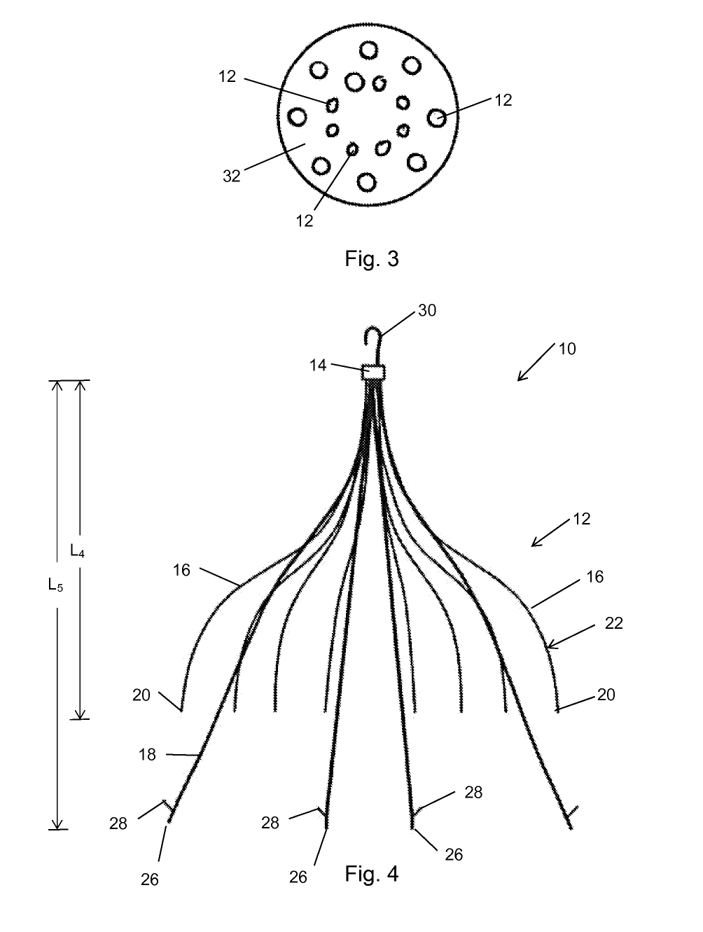

With reference also to FIG. 3, this shows a transverse cross-sectional view of the fastener element 32 close to its distal end 33, that is its end furthest from the hub 14. As can be seen, the legs 16 and 18 of the first and second sets of legs are embedded in circumferential arrays within the volume of biodegradable material forming the fastener element 32.

The fastener element 32 is made of a material which is substantially less flexible than the flexibility of the legs 16, 18 and is preferably of a material which is substantially rigid, such that the proximal ends 34 of the leg elements 12 are generally immovable within the fastener element 32. As the result, referring back to FIG. 1, the lengths of the legs 16 and 18 of the filter assembly 10 able to flex when the ends are embedded in the fastener element 28 are, respectively, L₂ and L₃, shown in FIG. 1. In other words, the legs 16 and 18 are only able to flex from the point beyond the end of the fastening element 32 to their tips or extremities 20 and 26.

The fastening element 32 is preferably formed of a material composition which will degrade after around two to three weeks after implantation into a patient's vasculature, although this time period may be shortened or extended as desired or necessary for the particular clinical application.

Referring now to FIG. 4, this shows the filter assembly 10 after the biodegradable faster element 32 has degraded, such that the proximal portions 34 of the leg elements 12 are no longer embedded within the fastener element. As a result, the legs 16 and 18 are able to flex along the entirety of their lengths, that is from the end of the coupling element or hub 14 to their distal ends 20 and 26, depicted as lengths L₄ and L₅, respectively, in FIG. 4.

A comparison between FIGS. 1 and 4 clearly shows the difference in the lengths of the legs 16, 18 which are able to flex when the fastening element 32 is and is not present. In the configuration of FIG. 1, with a shorter length of the legs 16, 18 able to flex, will produce a higher radial force upon a vessel wall, by virtue of the oversizing of the legs 16, 18, particularly at their points of maximum diameter, relative to the vessel in which the filter assembly 10 is deployed. Once the fastening element 32 has degraded, this will allow a greater length of the legs 16, 18 to flex, resulting in a reduction in the radial opening force generated by the legs 16, 18, and in particular at their points of maximum diameter (that is at points 20 and 16, respectively). This causes a reduction in the force exerted on the vessel walls.

In practice, it is important to have greater radial force produced by the filter assembly 10 on first implantation of the filter into a vessel, in order to reduce or eliminate the possibility of migration of the filter within the vessel. After around two to four weeks, typically, the portions of the filter legs 16, 18 which press against the vessel walls will become embedded into the vessel tissue as a result of tissue ingrowth, with the result that the ends will become fixed to the vessel wall. After this period, there is a risk of occurrence of later complications, such as ballooning or tenting of the vessel, stenosis of the vessel as a result of high radial forces produced by the filter, and/or of continued penetration of the ends of the legs 18 in particular into the vessel wall. However, with the structure disclosed herein on degradation of the fastening element 32 and the resultant increase in flexure length of the legs 16, 18, there is exhibited a reduction in the radial force produced by the legs 16, 18 impinging upon the vessel walls. This reduces the risks of later complications of the type mentioned.

In practice, the filter assembly 10 will be deployed endoluminally to the treatment site, typically through a catheter or sheath, with the legs 16, 18 radially compressed in the catheter and only able to expand radially outwardly once released from the introducer assembly, in a manner well known in the art. The fastener element 32 will not prevent the leg elements 12 from expanding radially outwardly, but will fix the proximal portions 34 of the leg elements 12 in position and as a result reduce the distance or length of the filter leg elements 12 which is able to flex. Once the bioabsorbable fastening element 32 has degraded, that fixation is lost and as a result that flexible length filter legs is increased in order to reduce the radial force produced by those filter legs on the vessel wall.

The structure ensures that the medical device 10 does not change its shape or configuration between the time when it is first implanted and after degradation of the fastener element 32. Thus, the shape of the medical device 10 remains steady and reliable in all its configurations. Moreover, the leg elements are preferably held in the fastener element with no tension applied to the legs or at best with only a small amount of tension.

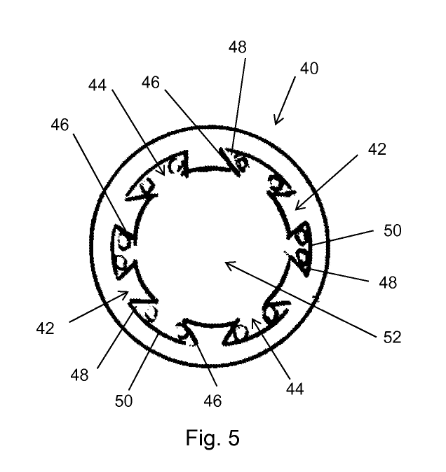

With reference now to FIG. 5, this shows another embodiment of fastener element 40 which can be used in place of the fastener element 32 of the embodiments of FIGS. 1 to 3.

The fastener element 40, which may be made of the same materials as the element 32, includes a structure having a plurality of radially inwardly extending flanges 42, which extend towards the radial centre line of the fastening element 40. The flanges 42 are spaced from one another to leave recesses 44 therebetween and have angled walls 46 which extend into the spaces of the recesses 44, thereby to create nooks or grooves 48 within which the leg elements 12 of the medical device 10 can be held. The structure of the internal surfaces of the fastener element 40 has a shape which could be described as being similar to that of a Maltese Cross.

The walls 50 of the recesses 44 and the impinging walls 46 create a fixation structure which fixes the first end of the leg elements 12 in position in the fastener element, in a manner which in practice is not dissimilar to when they are embedded within the fastener element as occurs in the embodiment of FIGS. 1 to 3. The first end of the leg elements 12 will be released only upon degradation of the fastener element 40, again in a manner similar to the embodiment of FIGS. 1 to 3. Once the fastener element 40 has degraded, the filter, in this embodiment, will have a structure as per the view of FIG. 4.

As will be appreciated, in the embodiment of FIG. 5, the fastener element 40 has a hollow interior lumen 52 which can be used for lining the passage of a guidewire through the medical device, such that this can be deployed over the wire. In other embodiments, there may be provided additional internal walls to the fastener element 40 in order to hold additional filter legs and in slightly different configurations, for example two sets of filter legs extending to different radial extents in a manner similar to the sets of legs in the depiction of FIG. 3.

The skilled person will appreciate that the number of recesses 48 provided in the fastener element 40 of the embodiment of FIG. 5 will be such as to match the number of filter legs. Furthermore, the skilled person will appreciate that the fastener element 40 may have a different cross-sectional view along the length thereof, with the arrangements of the walls 46, 50 of the structure being in alignment with the positions of the filter legs along the length of the first end of the legs, in order for these to be fixed in the deployed configuration. In the example of FIG. 4, therefore, the channels 44 and walls 48, 50 will taper radially inwardly towards the coupling member 14.

In the preferred embodiment, the bioabsorbable fastening element 32 degrades sufficiently to lose its binding strength on the proximal portions 34 of the leg elements 12 within two to three weeks (such that these can commence to flex more fully) and would be completely degraded within a period of one to two months.

In addition to generating a change in the radial opening force generated by the filter legs 16, 18, the use of a biodegradable fastening element 32 of the type disclosed herein also enables the use of filter leg materials which have reduced fatigue properties compared to those required for conventional filter assemblies, as a result of the lower forces which are subsequently generated by the filter assembly 10.

As will be appreciated from a consideration of FIG. 1 (and also of FIG. 4), two sets of filter legs 16 and 18 provides two sets of vessel contact points, being the points of maximum diameter of the filter legs 16 and of filter legs 18. These two sets of vessel contact points are spaced in the longitudinal direction of the filter assembly 10 and have the effect of stabilising the orientation of the filter assembly 10 in the vessel, preferably in order to keep the apex at the hub end 14 of the filter assembly 10 substantially aligned with the midpoint of the vessel. It is not excluded, though, that the filter assembly 10 may be provided with a single set of filter legs.

Although the preferred embodiments described herein have a set of filter legs (the legs 16) in which the legs are curved and a second set of filter legs (the legs 18) which are substantially straight, the shapes of the filter legs could be reversed relative to the embodiment shown and, similarly, both sets of filter legs 16 and 18 could be straight or curved.

In the embodiments described, the fastening element 32 is positioned in abutment with the coupling element or hub 14, which is the preferred arrangement, it is not excluded that the fastening element 32 could be spaced from the hub 14, further along the length of the leg elements 12.

The person skilled in the art will appreciate that the degradation times indicated above are exemplary only and that in practice the fastener element will be made of a material chosen to degrade within a time period deemed optimum for the particular vessel and medical condition to be treated. The skilled person will readily be able to determine these time periods and a suitable material composition on the basis of straightforward experimentation.

It will be appreciated that in the preferred embodiment the proximal ends 34 of the leg elements 16 and 18 are in their radially expanded configurations, that is their deployed configurations when embedded within the fastening element 32. It is not excluded that in some embodiments the proximal ends 34 of the leg elements 12 could be in orientations slightly different from their deployed orientations, for example, in a radially compressed configuration.

The coupling element or hub 14 preferably couples all of the leg elements 12 together, in one embodiment, as described above within a lumen of the hub 14. In other embodiments, some or all of the leg elements 12 may be laser cut from a common cannula which also forms the hub 14, in which case the legs are unitary with the hub, or at least some of these are so, with any remaining legs being fixed to the hub.

Although the embodiments described above show a single fastener element 32, the person skilled in the art will readily appreciate that other embodiments may have a plurality of fastener elements 32 and that these may have different degradation rates in order to create plurality of step changes in radial force produced by the leg elements 12 of the filter assembly.

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosure in the abstract accompanying this application is incorporated herein by reference.

1. An implantable medical device including:
a coupling member;
a plurality of leg elements made of flexible material, each leg element including a first end connected to the coupling member and a second end remote from the coupling member, each leg element having a length between the coupling member and its second end, at least the first end of the leg elements extending in a deployed configuration from the coupling member; and
a biodegradable fastener element, the plurality of leg elements being fixed in position in the fastener element along a portion of the leg elements adjacent the first end of the leg elements, wherein the fastener element substantially fixes said portions of the legs in the deployed configuration.

2. An implantable medical device according to claim 1, wherein the leg elements are embedded in the fastener element.

3. An implantable medical device according to claim 1, wherein the fastener element has a plurality of recesses within which the leg elements are held.

4. An implantable medical device according to claim 1, wherein the leg elements are fixed in the fastener element with substantially no tension applied by the fastener element to the leg elements.

5. An implantable medical device according to claim 1, wherein the fastener element is disposed immediately adjacent the coupling member.

6. An implantable medical device according to claim 1, wherein the fastener element is in abutment with the coupling member.

7. An implantable medical device according to claim 1, wherein the fastener element is attached to or integral with the coupling member.

8. An implantable medical device according to claim 1, wherein the fastener element is substantially rigid.

9. An implantable medical device according to claim 1, wherein the fastener element has a resiliency less than a resiliency of the leg elements.

10. An implantable medical device according to claim 1, wherein the fastener element reduces or prevents flexure of the portions of the leg elements fixed in the fastener element.

11. An implantable medical device according to claim 1, wherein degradation of the fastener element causes an increase in the length of the leg elements able to flex.

12. An implantable medical device according to claim 1, wherein the fastener element has a length at least 10 to 20% of the length of the leg elements.

13. An implantable medical device according to claim 1, wherein the fastener element has a length at least 10% of the length of the leg elements.

14. An implantable medical device according to claim 1, wherein the leg elements retain substantially the same non-biased configuration when partially embedded in the fastener element as on degradation of the fastener element.

15. An implantable medical device according to claim 1, wherein the first ends of the legs are rigidly attached to the coupling member.

16. An implantable medical device according to claim 1, wherein the fastener element is made of a biodegradable metal or metal alloy or of a biodegradable polymer.

17. An implantable medical device according to claim 1, wherein the device is a filter.

18. An implantable medical device according to claim 1, wherein the legs extend radially outwardly of the coupling member in a generally conical form.
